(19) United States
(12) Patent Application Publication
(10) Pub. No.: US 2008/0262866 A1
(43) Pub. Date: Oct. 23, 2008

(54) Methods for Improving the Clinical Outcome of Patient Care and for Reducing Overall Health Care Costs

(75) Inventor: Jeffrey C. Greene, Norman, OK (US)

Correspondence Address: TOMLINSON & O'CONNELL, P.C., TWO LEADERSHIP SQUARE, 211 NORTH ROBINSON, SUITE 450, OKLAHOMA CITY, OK, 73102, US

(73) Assignee: MedEncentive, LLC, Oklahoma City, OK (US)

(21) Appl. No.: 11/596,305

(22) PCT Filed: May 6, 2005
(86) PCT No.: PCT/US05/15791

§ 371 Date: Dec. 13, 2007

Related U.S. Application Data
(63) Continuation-in-part of application No. 10/841,240, filed on May 6, 2004.

(51) Int. Cl. G06Q 50/00

(52) U.S. Cl. 705/2

(57) ABSTRACT

The current invention is directed to improve methods for providing health care and for reducing the cost of health care. Additionally, the methods of the current invention are designed to improve the clinical outcome for the patient. The methods of the current invention provide financial incentives to both the patient and the medical service provider in an integrated manner that create appropriate checks and balances which encourages patient empowerment as well as patient and medical practitioner accountability. The method of the current invention incorporates evidence-based medicine treatment guidelines and content, and dispenses information therapy and other similar types of content through an Internet application or by other means to improve the standard of health care treatment which leads to better clinical outcomes, healthier behavior, and a reduction in the overall cost of health care.

### CROSS-REFERENCE TO RELATED APPLICATION

This application is a continuation-in-part of U.S. patent application Ser. No. 10/841,240 filed on May 6, 2004.

### BACKGROUND OF THE INVENTION

The current invention is directed to improvements in the delivery of health care. In particular, the current invention enhances the standard of care thereby providing to improved clinical outcomes and lowers overall medical costs. The current invention achieves these goals through the integration of evidence-based medicine (EBM) and information therapy (Ix) with a pay-for-performance (P4P) arrangement. The current invention utilizes Internet-based applications in order to improve the flow of information to health care providers and their patients. The current invention also utilizes a strategy of checks and balances to afford medical practitioners with the freedom to exercise clinical judgment, achieve medical practitioner and patient compliance, and prevent fraud and abuse.

Currently, the health care industry represents 15% of the country's GNP. Americans spend considerably more than citizens of any other developed countries on health care and yet Americans' life expectancy and infant mortality rates rank toward the bottom of these countries. A growing number of Americans are losing their health insurance because it is becoming increasingly unaffordable. Current estimates place the number of uninsured at 45,000,000. Fortune 500 companies have declared the current health care delivery system as unsustainable.

Since the mid-1980s, several attempts have been made to control the overall cost of the United States health care delivery system. Unfortunately, the attempted reforms only temporarily slowed the escalation of health care costs during the mid to late 1990s. More recently, health care costs have been rising at an alarming rate. In fact, based on currently available data, health care costs during 2001, 2002, 2003 and 2004 increased at about three to four times the rate of inflation. Therefore, a need exists for improved methods of delivering health care. Any health care reform should provide the means to control overall health care costs and should enhance the clinical outcome of medical treatment for the patient. In order to achieve these goals, the current invention provides a method of delivering health care that focuses on the parties having the greatest degree of control over costs and clinical outcome.

Studies by reputable organizations have concluded that the American health system is broken for the following reasons:
The healthcare in the United States is more expensive than any other developed country on earth and yet life expectancy and infant mortality in the U.S. ranks toward the bottom of developed countries.
45 million Americans are without health insurance coverage and this number continues to grow as coverage becomes increasingly unaffordable.
According to the RAND Corporation poor clinical outcomes and higher costs, result in part from patients receiving recommended care only 55% of the time.
Doctors and patients do not communicate well which results in misdiagnoses, poorer clinical outcomes and higher costs. A University of Toronto study determined:
Patients provide their medical practitioners with appropriate details only 2% of the time.
On average, medical practitioners interrupt their patients in the first 23 seconds of an encounter.
Patients understand what their medical practitioners tell them 15% of the time.
Patients follow their medical practitioner's instructions only 50% of the time.
The practice of defensive medicine by providers to reduce their malpractice risk adds 5% to 15% to the overall cost of healthcare.
The healthcare industry is one of the only industries where providers routinely get paid to fix their mistakes.
Medical practitioners and patients control the vast majority of cost (approximately 80%) and yet medical practitioners receive less of the premium dollar (17%) than pharmacy (22%), administration and underwriting (25%) and hospitals (28%).
Americans are increasingly unhealthy with preventable diseases such as obesity and diabetes reaching near epidemic rates.
Patients and medical practitioners are not accountable or empowered enough to improve health or control costs.
According to Boston University, inefficiencies in the American healthcare delivery system may account for 50% of the total cost of healthcare.

Additional studies and the consensus of opinion have concluded the following:
When the standard or quality of healthcare improves then clinical outcomes improve and overall costs are reduced. Therefore, the efficacy of a healthcare quality improvement program can be measured by cost trends.
The consensus of the medical community is that evidence-based medicine (EBM) treatments represent the highest standard of care. EBM is defined as “ . . . the conscientious, explicit, and judicious use of current best evidence in making decisions about the care of individual patients. The practice of evidence based medicine means integrating individual clinical expertise with the best available external clinical evidence from systematic research (Sackett D L, Rosenberg W M C, Gray J A M, Haynes R B, Richardson W S. Evidence based medicine: what it is and what it isn't. BMJ 1996; 312: 71-2).
Americans would prefer that their healthcare providers be compensated on the basis of value (which is referred to as pay-for-performance or P4P) as opposed to volume (Blue Cross and Blue Shield Association of America).
P4P programs have been successful in improving the standard of care and clinical outcomes.
According to the American Medical Association, P4P programs that mandate adherence to guidelines are considered by many medical practitioners as “cookbook medicine”, counterproductive, potentially dangerous, and will be rejected by the medical community.
Information therapy (Ix) changes patient behavior, improves clinical outcomes, and lowers costs (Blue Cross and Blue Shield Association of America and RAND Corp). Information therapy (Ix) is defined as dispensing the right clinical information, at the right time, so the patient can make the right decision about the management of their health. Proponents of Ix agree that it is powerful medicine.

According to experts, the success of P4P programs will hinge on:
medical practitioner (and patient) participation and compliance
the incorporation of evidence-based medicine (EBM);
the incorporation of information therapy (Ix);
investing first in quality improvement to achieve cost control;
the ability to effectively control fraud and abuse; and,
the cost of deploying and maintaining the program and the return on investment.

All of these factors contribute to the quality and cost of healthcare. Each factor must be taken into consideration to solve the problems that plague American healthcare. The developers of the current invention have done just that by creating a simple yet effective system that lowers healthcare costs by improving the standard of care.

### SUMMARY OF THE INVENTION

The current invention is designed to “bolt on” to health plans, including plans sponsored by health insurers, governments and self-insured employers, for the purpose of controlling costs. The current invention financially rewards medical practitioners and patients for controlling costs through utilization of evidence-based medicine treatment guidelines and information therapy.

The current invention provides a Program which financially rewards medical practitioners when they consider an evidence-based medicine (EBM) treatment guideline and prescribe information therapy (Ix) to their patients through a medical practitioner Internet Website application. These rewards are commonly referred to as pay-for-performance or P4P. This consideration of EBM and Ix is initiated as a result of the medical practitioner's normal filing of an insurance claim. In a preferred embodiment the process of the current invention takes place on a real-time basis. The current invention identifies applicable medical practitioner services from the filing of a claim for reimbursement and sends an e-mail notification to the medical practitioner. The medical practitioner responds to the e-mail through the medical practitioner portion of the Program Website. Appropriate responses by the medical practitioner will effect an automatic reimbursement increase to the medical practitioner and an Ix prescription to the patient.

When the patient receives the information therapy prescription by mail or e-mail, they are directed to a patient Website. There the patient is asked to read evidence-based medical content and answer a series of questions. These questions are designed to test the patient's understanding of their condition, determine their adherence to recommended treatment, and seek their impression of their medical practitioner's care relative to recommended care. As the patient answers these questions, they score points toward a financial reward or rebate of their out-of-pocket medical expenses. The patient's score is automatically forwarded to their Health Plan which provides a rebate of patient's costs. The current invention provides for the automatic or optional forwarding of the patient's actual responses to their medical practitioner to support subsequent care.

The current invention has a number of built-in features that are designed to achieve acceptance and produce better care while controlling costs. One of these features address medical practitioners concerns about being forced to practice “cookbook medicine.” The current invention allows, and in fact encourages, medical practitioners to deviate from treatment guidelines when it is appropriate in their judgment. The medical practitioner Website offers the medical practitioner a menu of reasons for a deviation or the medical practitioner can briefly describe the reason. The medical practitioner is eligible to be paid at the highest available rate if they indicate their adherence to or the reason for deviation from a guideline. This feature alleviates the concerns medical practitioners have about being forced to practice “cookbook medicine” and it communicates the medical practitioner's reason for deviation to their patients so they can understand that a particular guideline does not necessary fit their specific condition. This feature also helps the developers of guidelines determine which guidelines are strongest and which ones need further research and development.

In a preferred embodiment, the current invention separates the financial reward provided to the medical practitioner from the reward provided to the patient. Thus, the medical practitioner is paid for their time and effort independent of how their patient responds to their information therapy or adherence to treatment. On the other hand, the patient's financial reward is dependent upon their medical practitioner prescribing information therapy and upon the patient's adherence to self-managing their health. The strategies for providing rewards have been purposely configured to create a natural and beneficial check and balance between the medical practitioner and the patient. This set of strategic checks and balances solves the issues of compliance monitoring and appropriate provider deviation from a guideline that other P4P models cannot solve.

The current invention provides a method for delivering health care services designed to lower health care costs and improve patient clinical outcomes by elevating the standard of care and encouraging patients to lead healthier lives. The method comprises the steps of receiving a claim for compensation for medical services from a medical practitioner for medical treatment of a patient. The claim includes at least one applicable diagnosis code corresponding to at least one applicable medical treatment received by the patient. If at least one diagnosis code in the submitted claim corresponds to a medical diagnosis found in a data base of applicable medical diagnoses, then a notice is sent to the medical practitioner providing the medical practitioner access to a website. The website provides the medical practitioner with access to EBM treatment guidelines or other pertinent medical content relating to the medical diagnosis. In addition to the common medical treatment, the medical practitioner prescribes information therapy for the patient. The Ix provides the patient with instructions concerning managing the medical condition and living a healthy lifestyle. Subsequently, the medical practitioner rates patient compliance with the prescribed information therapy and instructions relating to a healthy lifestyle.

In another embodiment, the current invention provides a method for delivering health care services. The method of the current invention comprises the steps of receiving a claim for compensation for medical services from a medical practitioner for medical treatment of a patient. The claim presented by the medical practitioner includes at least one applicable diagnosis code corresponding to at least one applicable medical treatment rendered to the patient. Upon receipt, the claim is examined to determine if at least one diagnosis code corresponds to an applicable medical diagnosis found in a data base of applicable medical diagnoses. If a corresponding applicable medical diagnosis is present, then a notice is sent to the medical practitioner. The notice sent to the medical practitioner includes the instructions necessary for accessing a website. Once the medical practitioner gains access to the website, the medical practitioner will have access to EBM treatment guidelines relating to the medical diagnosis. Thereafter, the medical practitioner prescribes information therapy for the patient. The prescribed information therapy includes guidelines for healthy behavior. Additionally, the medical practitioner rates patient compliance with the prescribed Ix and recommended health maintenance. Following prescription of Ix, the website automatically generates a notice to be provided to the patient directing the patient to access the website. Once the patient accesses the website, the website will provide the patient with the means to access medical information relating to the medical diagnosis. The method further provides for the monitoring of the patient's access of the medical information. The website also provides a knowledge exam designed to measure patient comprehension of the medical diagnosis and the prescribed Ix. Provided that the patient takes the exam, the website will automatically score the knowledge exam and it will provide the patient with the option of forwarding the knowledge exam results to patient's Health Plan. Finally, the patient is provided with the option of authorizing transmission of the compliance rating assigned by the medical practitioner to the patient's Health Plan and/or employer for the purpose of determining a financial reward.

In a further embodiment of the current invention, the patient is provided with the option of rating the medical practitioner's compliance with EBM treatment guidelines or other accepted treatment guidelines corresponding to the medical practitioner's diagnosis. In this embodiment of the invention, the patient is advised by the patient portion of the website of a deviation from the EBM or other accepted treatment guidelines and provided with the medical practitioner's reason for the deviation. Following review of the prescribed Ix material, the patient is asked to rate the medical practitioner's judgment against medical science and to provide a performance rating. This rating does not directly affect the medical practitioner's reward or rewards on a case-by-case basis. However, it does begin to build an overall clinical performance rating for that medical practitioner. This can be used to help individual medical practitioners measure their performance against their peers. Poor ratings can be used in peer review. This embodiment of the invention allows and encourages medical practitioners the freedom to use their clinical judgment to deviate from a guideline while receiving the maximum financial reward, provided the medical practitioner selects or supplies a reason for the deviation. Preferably, the ratings provided by the medical practitioner and the patient would be obscured from the other party to help protect the doctor-patient relationship with each party having the option of releasing their rating.

Still further, the current invention provides a system for delivering health care services. The system of the current invention comprises a website having a medical practitioner portion and a patient portion. The medical practitioner's portion is programmed to receive a coded claim for medical services rendered by the medical practitioner. The website compares the coded claim to a database of medical diagnoses, if the coded claim matches a medical diagnosis in the database, then the website sends a notice to the medical practitioner directing the medical practitioner to access the website. The medical practitioner portion of the website includes a database of EBM guidelines, patient names, dates of services provided and medical diagnoses accessible by the medical practitioner. The website provides the medical practitioner with the ability to indicate compliance or noncompliance with EBM guidelines for the medical treatment provided. Additionally, the website provides for input by the medical practitioner of a patient's compliance with prescribed treatment. Further, the website tracks the medical practitioner's usage of the website. The patient portion of the website includes a database containing at least the Ix relevant to the patient's condition and medical treatment provided by the medical practitioner. The website is accessible by the patient and the website provides the patient with a knowledge exam designed to assess the patient's understanding of the Ix and prescribed medical treatments. The patient portion of the website monitors the patient's access of the website and scores the knowledge exam. Additionally, the patient portion of the website provides the patient with the ability to forward the results of the knowledge exam to a third party. Typically, the third party will be the patient's employer and/or insurance provider. The patient portion of the website provides the patient the option of authorizing the release of their medical practitioner's assessment of patient compliance with prescribed treatment to a third party. Finally, the patient portion of the website provides the patient with the ability to review the medical practitioner's reason for any deviation from EBM and other accepted treatment guidelines for the diagnosed condition. The patient portion of the website further provides the patient with the ability to rate the treatment provided by the medical practitioner in view of the EBM and other accepted treatment guidelines.

### BRIEF DESCRIPTION OF THE DRAWINGS

FIG. 1 is a flow chart representing the medical practitioner's portion of the Program.

FIG. 2 is a flow chart representing the patient's portion of the Program.

FIG. 3 is a flow chart depicting one embodiment of the current invention.

FIG. 4 is a flow chart depicting the operation of the current invention as a real-time system.

FIG. 5 is a second flow chart depicting the operation of the current invention as a real-time system.

### DETAILED DISCLOSURE OF THE PREFERRED EMBODIMENTS OF THE CURRENT INVENTION

The current invention provides methods for improving the standard of care while simultaneously lowering overall health care costs. The improved health care delivery methods achieve the desired results by focusing on those parties having the greatest control over health care costs, i.e. the medical practitioner and the patient. In particular the current invention integrates the roles of the patient and medical practitioner in determining the medical treatment.

In one embodiment the current invention provides a Program for delivering health care according to the following outline:

### Patient Seeks a Medical Practitioner:

When an Employee/Insured or their covered-dependents (collectively referred to as the “patient”) seek healthcare, they refer to a printed or Web-based directory of medical practitioners.

### Patient Notifies Their Doctor of Coverage in a Health Plan Using the Current Invention:

Patients are encouraged to mention they are covered by the Program of the current invention when they make an appointment, when they present themselves to their medical practitioner's front desk and when they greet their medical practitioner. The patient is also encouraged to talk with their medical practitioner about Evidenced-Based Medicine, Information Therapy, and the financial rewards administered through the Program of the current invention.

### Medical Practitioner Acknowledges Participation and Can Access Evidence Based Medicine Content and Ix Content During Patient Encounter:

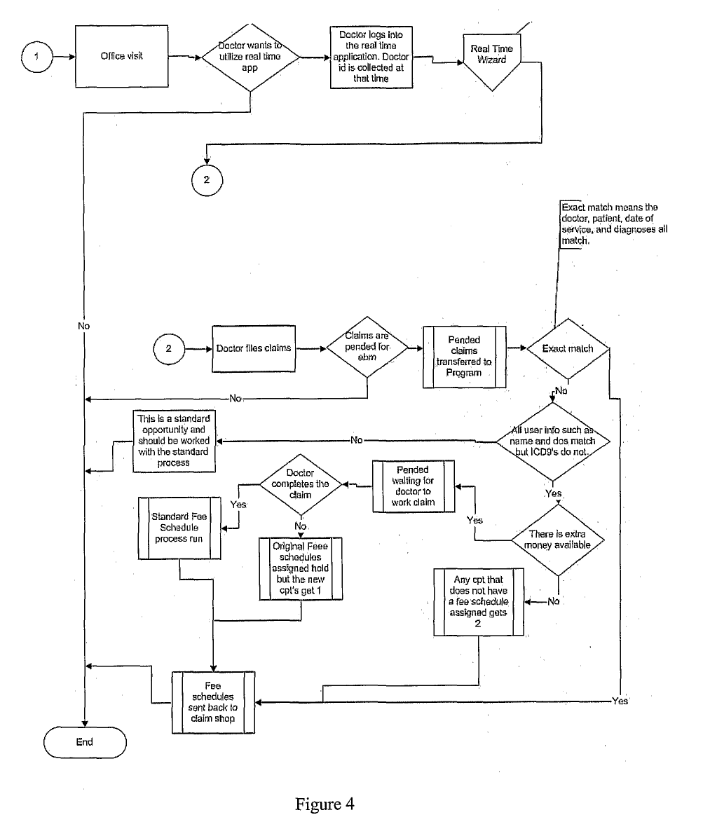

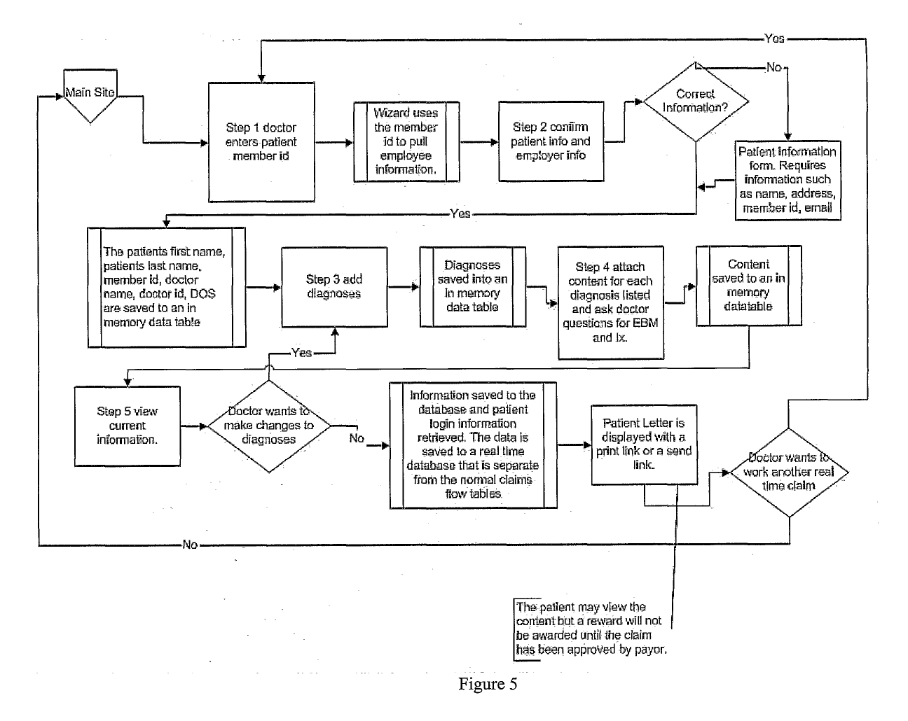

If the medical practitioner is familiar with the Program of the current invention, they may choose to initiate the pay-for-performance Opportunity and retrieve evidence-based treatment guidelines through the Program Website during the patient encounter, or they can wait until the Program Website responds to the medical practitioner's filing of the patient medical insurance claim with a pay-for-performance notification. Thus, the Program may be initiated on a real-time basis while the medical services are being rendered. This is accomplished when the medical practitioner accesses the Program website and enters pertinent information such as patient identification and diagnosis(es). As depicted in FIGS. 4 and 5, the real-time version of the Program searches its data base to determine if the patient is eligible and/or to find an applicable diagnosis(es). If the Program's computer identifies a match, then it retrieves available EBM treatment guidelines and other pertinent information related to the diagnosis(es). The Program Website subsequently presents the available guideline(s) and other pertinent medical content and asks two questions: “Are you following this guideline in the treatment of this patient?” and “Do you wish to prescribe Information Therapy to this patient?” Optionally, the Program may ask the following: “Indicate this patient's compliance to recommended care for this diagnosis.” If the medical practitioner answers yes to these two questions and, optionally, the medical practitioner indicates the patient's compliance to recommended care, then the Program Website automatically selects the highest available rate of payment for the medical practitioner's associated services. If a guideline does not exist for a given diagnosis, then Ix content is made available to prescribe, for which the medical practitioner receives a slightly lower or intermediate level of compensation. These responses made by the medical practitioner on the Website are stored in the Program's data base until they are matched to the corresponding claim for reimbursement submitted by the medical practitioner
   Medical Practitioner Files Medical Insurance Claim and Receives a pay-for-performance Notification E-mail Response or Initiates an Automatic Determination of Reimbursement Rate:
When the medical practitioner files a covered patient's health insurance claims, the Program Website application keys off the claim's diagnosis and treatment information to automatically send back an email asking the medical practitioner to consider an EBM treatment guideline and an Ix prescription to the patient. The medical practitioner must file a claim within specific time limits. If the medical practitioner fails to file their claim within these time limits, then they will miss the opportunity to earn a higher rate of reimbursement. If the medical practitioner initiates the pay-for-performance opportunity at the time-of-service through the real time version of the Program Website, then the pay-for-performance notification will be generated at that time without the claim filing process. The medical practitioner's responses to the Website's queries are stored in the Program's data base until a claim is sent. When the claim for reimbursement is submitted by the medical practitioner and the claim's information such as the medical practitioner identification, patient identification, date of service, and at least one applicable diagnoses linked to at least one applicable medical service matches information supplied by the medical practitioner through the Program's real-time version and stored in the Program's computer, then the Program automatically determines the medical practitioner's rate of reimbursement and no email notification is sent to the medical practitioner for purpose of determining reimbursement rate. If a submitted claim does not match stored information from the Program's real-time version or if a submitted claim contains information that the Program's computer determines could further increase the medical practitioner's rate of reimbursement, then an email notification containing a pay-for-performance opportunity is sent to the medical practitioner.
   Medical Practitioner Earns a Financial Reward by Responding to pay-for-performance Notifications:
When the medical practitioner answers the email, eligible claims will appear. When the medical practitioner clicks on a patient claim, the Program Website presents an evidence-based decision-tree treatment guideline(s) and other pertinent medical content and asks two questions: “Are you following this guideline in the treatment of this patient?” and “Do you wish to prescribe Information Therapy to this patient?” Optionally, the Program may ask the following: “Indicate this patient's compliance to recommended care for this diagnosis.” If the medical practitioner answers yes to these two questions and, optionally, the medical practitioner indicates the patient's compliance to recommended care, then the Program Website automatically selects the highest available rate of payment for the medical practitioner's associated services. If a guideline does not exist for a given diagnosis, then Ix content is made available to prescribe, for which the medical practitioner receives a slightly lower or intermediate level of compensation. If the medical practitioner chooses not to participate or fails to respond within a time limit, then they are paid at a third and lowest level of compensation for the related services. There are time limits for the medical practitioner to respond to pay-for-performance notifications. If these time limits are exceeded then the medical practitioner will fail to earn a higher rate of pay. However, the medical practitioner may still be able to initiate an Information Therapy prescription, thus preventing penalizing the patient. If the medical practitioner initiates the pay-for-performance opportunity at the time-of-service through the Program Website in the real-time version of the Program, then the responses to the two or three queries described in this step are stored in the Program's data base. When the medical practitioner submits a claim for reimbursement for these medical services, then the Program matches the claim information with the stored responses and automatically determines the medical practitioner's rate of reimbursement. The email notification to the medical practitioner is not needed in this embodiment of the invention.
   Medical Practitioner Can Deviate from a Treatment Guideline:
The Program allows the medical practitioner to deviate from a treatment guideline if the guideline is not appropriate for a particular patient or medical condition. If a medical practitioner answers “no” to the guideline adherence question, then a menu of appropriate reasons for deviation from a guideline pops-up. When the medical practitioner selects and responds to a valid reason for deviating, the current invention automatically selects the highest available rate of payment for the medical practitioner's associated services, just the same as if the medical practitioner had answered “yes” to this question.

### Transmission of an Information Therapy Prescription to the Patient:

When a medical practitioner selects a “yes” response to the Information Therapy question, the current invention automatically prints and sends an Ix prescription notice to the patient. If a patient is pre-registered in the Program, the preferred notice is an e-mail. If the patient receives their Ix prescription at the time-of-service in the real-time version of the Program, then there is no need to send the patient the prescription after-the-fact in this embodiment of the invention.

### Patient Responds to Information Therapy Prescription:

The notice explains its purpose and directs the patient to the Program Website. The letter or e-mail also contains a user identification number and passcode to allow the patient access to their Ix. If the medical practitioner is initiating the pay-for-performance opportunity at the time-of-service on a real time basis, then the patient may receive their Ix prescription before they leave the medical practitioner's office. Whether the patient receives their Information Therapy prescription by letter, e-mail or in the office at the time-of-service, the patient must respond within a time limit. If the patient fails to respond, then the current invention may initiate an automatic telephone call reminding the patient to access the Program Website. If the patient still does not respond, then they will lose the opportunity to gain valuable information about their health and fail to earn a financial reward.

### Patient Reads Information Therapy Content and Answers a Series of Pertinent Questions:

When the patient accesses the Program Website, they are asked to read information about their medical condition and answer questions that test their understanding of the information. They are also asked about their medical practitioner's adherence to the recommended treatment and their own adherence to the recommended treatment. In the process, the patient learns how to best manage their health and encourages them to do so. This feature creates a natural and strategic check and balance between the patient and medical practitioner that helps improve the standard of care, encourage health behavior, control healthcare consumption, and prevent fraud and abuse.

### Patient's Answers to the Questions Earn Them Points Toward a Financial Reward:

The patient is asked to successfully accomplish the following tasks: 1) read the information presented to them in the Program's Website about their health condition and recommended (EBM) and appropriate care, and answer questions to demonstrate their understanding of this information; 2) indicate their compliance to the recommended (EBM) and appropriate care which may include reporting health status such as weight, blood pressure, blood sugar, and resting heart rate or may request authorization to access pharmacy records to verify that prescriptions have been filled or may request verification that the patient has successfully participated in a health assessment program or may request verification that the patient has seen a medical specialist or successfully completed other recommended therapies; 3) after acknowledging their medical practitioner's adherence to or deviation from a recommended treatment and taking into consideration the information they have just read in the Program's Website, rate their medical practitioner's performance; and optionally, 4) elect to have their medical practitioner's rating of their compliance to recommended and appropriate care included in the calculation of their financial reward or health status. This election by the patient reinforces the Program's strategic checks and balances which rely on the patient honestly indicating their personal compliance to recommended care, appropriate self-management and a healthy lifestyle. The patient is aware that this election will cause the Program to compare their personal health compliance responses against their medical practitioner's rating of their health compliance. If the compliance indicators between the patient and the medical practitioner match, then the Program would indicate that the patient is eligible for an additional financial reward from their Health Plan. When the patient appropriately answers the questions on the Program Website, they earn points. Once the Patient's point total achieves a pre-determined level, a notice pops-up advising them that they are eligible for a financial reward. The current invention automatically forwards the patient's score to their employer or health plan who can affect the financial reward.

### Patient's Responses are E-mailed to Their Medical Practitioner to Help Improve the Standard of Care:

The patient's actual responses are emailed to their medical practitioner so that they can be added to the patient's medical chart. This information can then be used to help the medical practitioner stay abreast of the patient's on-going status and manage the Patient's subsequent care. The feature also creates another strategic check and balance.

Preferably, the system of the current invention will provide suitable incentives to both the patient and the medical provider to bring about a change in behaviors resulting in better clinical outcomes for the patient while lowering overall costs for the health care system. Additionally, the improved method for delivering health care aligns the interests of all the key stakeholders in the health care industry. These key stakeholders are generally identified as medical practitioners, patients, employers, insurers and hospitals. For the purposes of this discussion, the current invention focuses on services delivered by a medical practitioner such as a physician; however, the methods of the current invention apply equally well to all medical service providers. The health care delivery system referred to herein as the Program will improve the standard of care and lower costs.

The current invention provides these benefits by financially rewarding medical practitioners and patients through a “pay-for-performance” or “P4P” arrangement. In the method of the current invention, patients and medical practitioners receive financial rewards when they voluntarily follow evidence-based medicine (EBM) guidelines and appropriately respond to questions relating to EBM and medical practitioner prescribed treatment including instructions relating to living a healthy lifestyle and managing the medical condition. Through use of “Information Therapy (Ix),” the current invention empowers the patient, allowing the patient to take an active role in managing their own health care, thereby improving their overall quality of life. The combination of EBM and Ix will improve the standard of care and lower overall healthcare costs.

To encourage medical practitioner participation in the method of the current invention, medical practitioners will be financially rewarded for each patient encounter when the medical practitioner accomplishes the following tasks for each treated diagnosis: 1) if available, consider EBM and other recommended treatment guidelines and indicate adherence to the guideline or deviation from the guideline for an appropriate reason that can or will be communicated to their patient through the Program website, 2) prescribe information therapy to their patient (not optional for a financial reward), and 3) rate the patient compliance to recommended care for each diagnosis.

Medical practitioners have the freedom to indicate their adherence to an EBM guideline or recommended treatment or to indicate their deviation from the guideline or recommended treatment for an appropriate reason supplied by the medical practitioner. The option to deviate from a guideline is offered to medical practitioners because their patients are subsequently asked to rate their medical practitioner's performance against the EBM or recommended treatment guideline during the patient's Ix session. The patient's rating may be used to affect the medical practitioner's compensation directly or may be used to rate the medical practitioner against his/her peers or against other standards in an effort to improve performance or remove the medical practitioner from a health plan for gross aberrancy after due process. Thus, the method of the current invention provides medical practitioners with the freedom to adhere or deviate from a guideline based on their sound clinical judgment while still being rewarded financially. In exchange for the right to practice medicine without rigid guidelines, the medical practitioner allows the patient to review the prescribed treatment in view of the recommended or EBM treatment guidelines.

As an encouragement to respond to Ix prescriptions and to live a healthy lifestyle, the methods of the current invention enhances the financial outcome for the patient when the patient completes the following tasks: 1) read the information presented to them on the methods webpages about their health condition and recommended (EBM) care, and answer questions to demonstrate their understanding of this information; 2) indicate their compliance to the recommended (EBM) and appropriate care which may include reporting health status such as weight, blood pressure, blood sugar, and resting heart rate or may request authorization to access pharmacy records to verify that prescriptions have been filled or may request verification that the patient has successfully participated in a health assessment program or may request verification that the patient has seen a medical specialist or successfully completed other recommended therapies; and, 3) after acknowledging their medical practitioner's adherence or deviation from a recommended treatment and taking into consideration the information they have just read on the Program website, rate their medical practitioner's performance; and optionally, 4) elect to have their medical practitioner's rating of their compliance to recommended and appropriate care included to calculate their financial reward or health status.

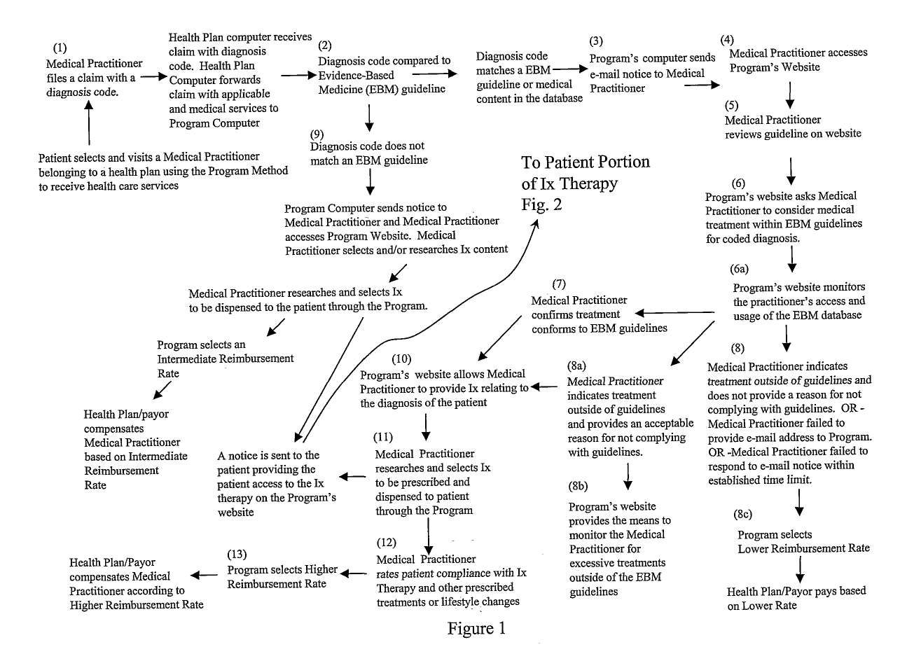

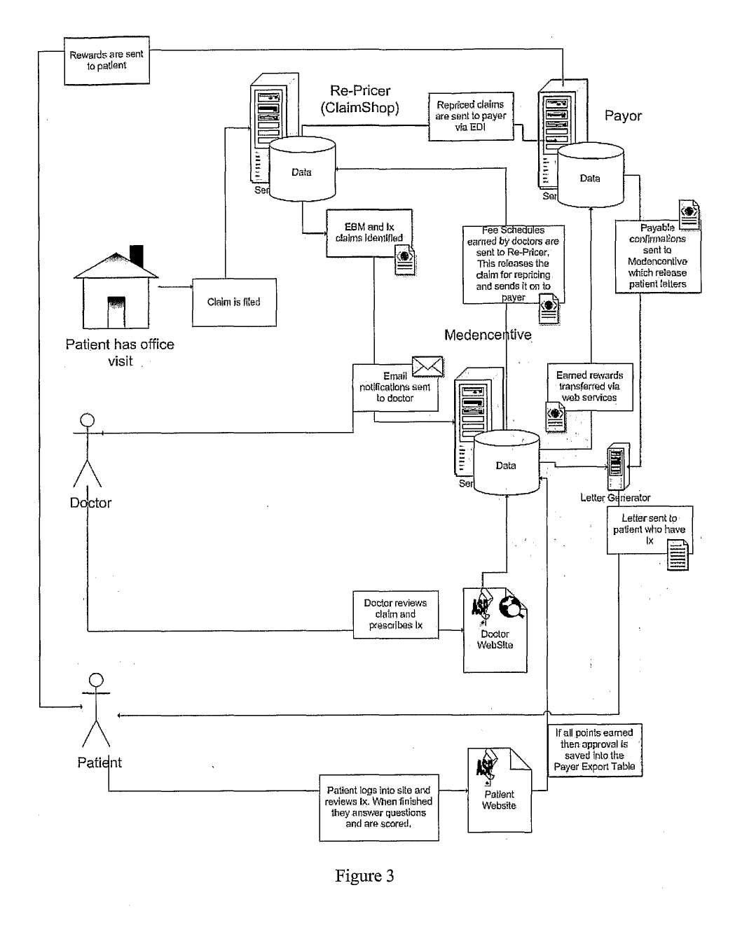

The health care delivery methods of the current invention will be described with reference to FIGS. 1-3. To aid in identification of the various steps of the current invention, identifying numbers are provided for selected portions of the process. Electronic communications, such as but not limited to the Internet and e-mail, provide the most efficient means for practicing the methods of the current invention. However, the methods of the current invention may be readily adapted to traditional mail, faxes and other hard copy communications or a blend of electronic communication and traditional communications.

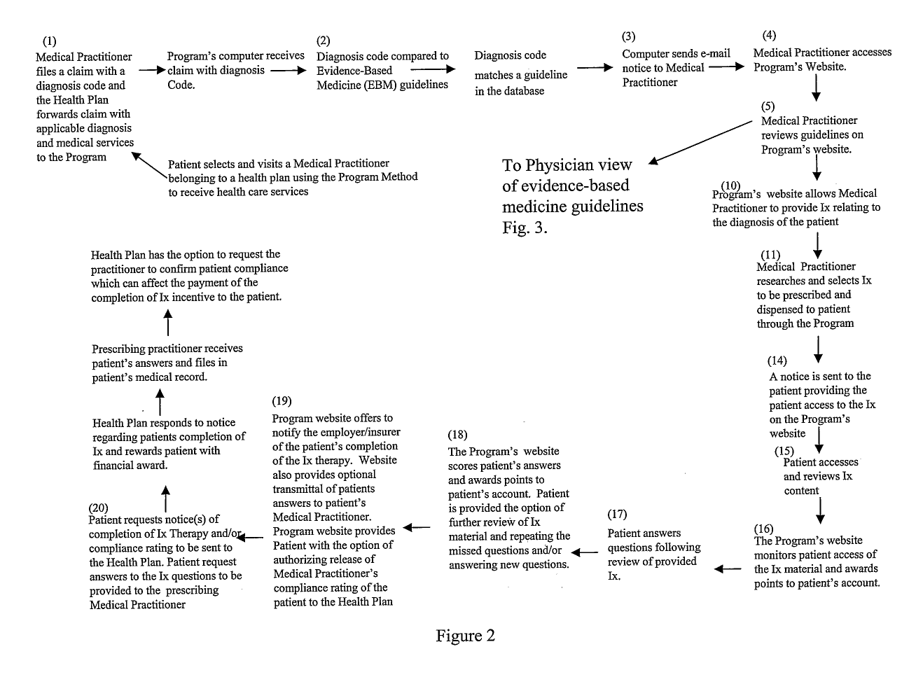

FIGS. 1-3 provide flow charts of preferred embodiments of the method for providing health care. FIG. 1 outlines a preferred embodiment of the current invention as it relates to the medical practitioner's portion of EBM and Ix Therapy. FIG. 2 outlines the patient's portion of a preferred embodiment of the current invention. While shown in step wise format, those skilled in the art will recognize that various portions of the process can be moved earlier and later in the charts. The methods of the current invention are designed to provide flexibility and adaptability depending on the desires of the local health plan. The format of the current invention may be adapted by any form of health plan. Fee-for-service PPO's and governmental programs are particularly suited for performing the methods of the current invention. As used herein, the term “Health Plan” refers to the organization managing the health care delivery system and may include any payor type system including a self-insured employer, health insurance companies, managed care plans, and governmental programs such as Medicare, military, state and Federal employees, and Indian Health Service.

As shown in FIG. 1, the method of the current invention begins with educating the patient and the medical practitioner on why and how the methods of the current invention (referred to herein as “the Program”) work. Prior to receiving treatment, the patient identifies a medical practitioner that participates in the Program. Typically, the Program will be administered or carried out by a health plan. The patient presents to the participating medical practitioner as a member of the Program, incorporating EBM, Ix, and the financial reward arrangement. Subsequently, the medical practitioner provides health care services to the patient.

Preferably, the medical practitioner accesses the Program's website at the time of service and enters pertinent patient information and diagnosis(es) information preferably as a standardized diagnosis(es) code(s). The Program's software application compares the patient and diagnosis(es) information to the Program's data base. If the Program's software finds a patient information match in the Program's data base and there is available EBM or recommended treatment guidelines and Ix content related to the diagnosis(es) in the data base, then the Program displays the guideline and/or Ix content to the medical practitioner on the website. The website is interactive. As such, if an EBM or recommended treatment guideline is available, the medical practitioner considers the guideline and indicates adherence to the guideline or provides a reason for deviation from the guideline on the website. If Ix content is available, the medical practitioner selects or searches for the preferred Ix content and orders the Ix prescription to the patient on the website. Optionally, the medical practitioner is asked to rate the patient's compliance to EBM or appropriate care for each presenting diagnosis. The patient and diagnosis(es) information, the medical practitioner's response(s) to guideline adherence, the Ix prescription order, and the medical practitioner's rating of the patient's compliance are stored in the Program's data base for subsequent processing to determine the medical practitioner's rate of reimbursement.

The Ix prescription can be made available to the medical practitioner at the time of service so it can be printed and handed to the patient or it can be mailed or e-mailed to the patient. Alternatively, the medical practitioner may choose to postpone reviewing guidelines and prescribing Ix until after a claim for reimbursement is filed. Therefore the process can be initiated at the time of service by the medical practitioner accessing the Program's website or it can be initiated by filing a claim for reimbursement.

Following treatment of the patient, the medical practitioner files a claim for reimbursement with the Health Plan administering the Program. Preferably, the medical practitioner files the claim electronically (1). The medical claim contains information commonly found on current claim forms such as the patient's name, the medical practitioner's name, a primary medical diagnosis, secondary diagnosis(es) and the service provided by the medical practitioner. Preferably, the medical diagnosis and the medical services are identified by a usual and customary diagnosis medical services codes, and the diagnosis(es) is appropriately linked to the corresponding medical service(s).

Upon receipt of the claim, patient and diagnoses information are compared to any matching information in the Program's data base from the time of service. Matches then determine if the claim lists medical services eligible for a variable rate of reimbursement (referred to as “applicable medical service(s)”) contained in the Program data base (2). If the claim contains applicable medical services, then the medical practitioner's stored responses to queries concerning guideline adherence, Ix prescription, and patient compliance for the diagnosis(es) linked to the applicable medical services are taken into consideration in determining the medical practitioner's rate of reimbursement as described herein.

Practitioners must submit a claim for reimbursement within a time limit or they will not be eligible for the higher rates of reimbursement. If information supplied by the medical practitioner at time of service is not matched to a claim within a certain period of time, then the Program may send a warning to the medical practitioner that the claim filing time limit is running out.

Alternatively, if the medical practitioner did not access the website or respond to the website queries at the time of service, then the Program's computer will not identify matching patient and diagnosis information. If this is the case, then the computer the compares the claim information to the Program's data base for applicable diagnoses. If the claim contains an applicable diagnosis, then the computer determines if the diagnosis is linked to an applicable medical service. If this is the case, then the medical practitioner receives a notification informing the medical practitioner that EBM guidelines and Ix content are available for review (3). In the preferred embodiment, a practitioner will file the claim electronically with the diagnosis and corresponding medical service(s) identified by usual and customary codes.

In a preferred embodiment, a computer capable of comparing the diagnosis and medical service(s) codes to the Program data base receives the claim. If the encoded diagnosis and corresponding medical service(s) matches the code for medical diagnosis and applicable medical services within the Program data base, then the computer automatically transmits an email, fax or other electronic correspondence to the medical practitioner, or the computer system prepares a regular letter for mailing to the medical practitioner.

The notification sent to the medical practitioner advises the medical practitioner to access the medical practitioner's portion of the Program's website containing EBM guidelines or other health care quality improvement and cost control methods. The Program website is preferably a secure website requiring input of the medical practitioner's password to gain access to the data contained therein. Alternatively, these codes may be transmitted by a separate email or otherwise provided to the medical practitioner. The method for gaining access to the website is not critical to the current invention.

For the purposes of this disclosure the term website refers to the Program's website. The Program's website may or may not be located on a central server. Further, the patient and practitioner portions of the Program's website are not necessarily contained on the same computer system. As used herein, the medical practitioner portion of the Program's website will preferably be utilized by all parties authorized to access the medical practitioner's portion of the website including but not limited to nurses, nurse practitioners, physician assistants and other care providers.

Upon entry of the appropriate codes or passwords at the website (4), the website identifies the names of patients, the dates and types of services provided, the medical diagnoses and related medical services for the accessing practitioner or their authorized assistant. The website also provides the available EBM guidelines or other health care quality improvement and cost control methods corresponding to each diagnosis. Preferably, the medical practitioner reviews and confirms the appropriateness of the information found on the website (5).

The Program's website is interactive. As such, it queries the medical practitioner concerning adherence to EBM guidelines or other health care quality improvement and cost control methods for the diagnoses (6), the prescription of Ix to the patient, and patient compliance with the prescribed treatment and guidelines on living a health lifestyle and methods for controlling/managing the patient's medical condition (12). The medical practitioner's response to the queries will determine the reimbursement rate used to compensate the medical practitioner for services rendered on each claim. If the medical practitioner responds to the query concerning patient compliance, confirms the prescription of Ix to the patient, and confirms treatment within the scope of the EBM guidelines or other health care quality improvement and cost control methods or provides appropriate reason(s) for deviation from guidelines and methods (7), then the website will automatically direct compensation to be made according to a higher payment (practitioner reimbursement) rate scale (13). Preferably, the highest payment is selected only after the medical practitioner has indicated adherence to or deviation from, with appropriate reason, a recommended treatment guideline, prescribed Ix for the patient (10, 11) and has rated patient compliance (12). A secondary level of payment is selected when the medical practitioner has prescribed Ix for the patient and has rated patient compliance, but no treatment guideline is available.

As noted above, the website also queries the medical practitioner concerning the patient's compliance with EBM guidelines, Ix and any lifestyle activities necessary to improve the patient's wellness. Preferably, the website will provide the medical practitioner with the opportunity to rate patient compliance (12) with the recommended treatment and behaviors using the following terms: Compliant, Partially Compliant, Non-compliant and Non-applicable. Alternatively, the patient compliance rating terms may be: Compliant and No Response. No Response may mean partially compliant, non-compliant, or non-applicable. To receive the highest compensation level (13) for the services provided, the medical practitioner may need to respond to the request for a patient compliance rating. The ratings provided by the medical practitioner will be stored by the Program website awaiting a response by the patient to the prescribed Ix. However, the patient will not have the ability to see the medical practitioner's rating unless the medical practitioner has selected the option to permit patient viewing of the rating.

Typically, the medical practitioner must access the interactive website within 48 to 72 hours of receipt of the notification in order to qualify for the higher payment rate scale. In the preferred embodiment, the medical practitioner is required to respond to the notice within 48 hours or two business days. If the medical practitioner does not respond within the indicated period of time (8), then the website will direct compensation to be made according to a lower rate scale.

As previously indicated, the Program's website is interactive. To provide the maximum flexibility and greatest possibility of improved clinical outcome for the patient, the method of the current invention does not rigidly limit the medical practitioner only to the EBM guidelines in order to receive the highest degree of compensation. Rather, the Program's website provides the medical practitioner with the option of indicating the treatment falls outside of the guidelines while explaining the reason for prescribing treatment outside of the guidelines. Provided that the medical practitioner completes the section describing an appropriate reason for non-adherent treatment (8a), the Program's website will still select the highest compensation level for the medical practitioner (13). Thus, the present invention avoids the practice of “cookbook medicine” by encouraging the medical practitioner to use appropriate judgment and medical skill when deciding to follow the EBM guidelines or choosing to deviate from the guidelines. As previously indicated, in the preferred method the medical practitioner must prescribe Ix for the patient and rate patient compliance with directions/guidelines on living a health lifestyle and methods for controlling/managing the medical condition (12) before becoming eligible to receive payment at the highest or second highest (intermediate) compensation rates.

While the medical practitioner is not required to indicate compliance with the EBM guidelines, failure to respond within 48 hours or indicating non-adherence without providing an appropriate reason for treatment outside of the EBM guidelines will have a negative financial impact on the medical practitioner. Specifically, these actions will trigger the computer system to select the lowest possible payment scale for the medical practitioner's services (8c). If the medical practitioner fails to prescribe Ix for the patient, then the website will direct the selection of the lowest payment scale for compensation of the medical practitioner.

Thus, the method of the current system provides a financial incentive to the medical practitioner to follow the EBM guidelines or to provide an appropriate reason for deviating from these guidelines. Additionally, the method of the current invention provides a financial incentive to the medical practitioner to prescribe Ix to the patient and to rate patient compliance with the prescribed treatment/lifestyle necessary to manage the medical condition (12). In general, treatment according to the EBM guidelines and appropriate treatment outside of the guidelines coupled with patient compliance with treatment protocols and a healthy lifestyle will produce better clinical outcomes for the patient. Further, the prescription of Ix to the patient empowers the patient to be more compliant with their medical practitioner's treatment orders and instructions leading to improved clinical outcomes. Additionally, the patient's access to Ix provides the patient with the tools to control the medical condition thereby reducing medical practitioner visits, the need for pharmacy and other therapies, and expensive hospitalizations. Thus, the current invention provides a method for improving clinical outcome and for reducing health care costs. Clearly, the current invention integrates the activities of the patient and medical practitioner by encouraging the incorporation of EBM and Ix with financial incentives.

In order to provide practitioner compliance and to prevent fraud and abuse, the Program's website provides the means to monitor and audit the medical practitioner. In one aspect, the website provides the means for tracking the medical practitioner's access to the website. This tracking mechanism provides an indication of the medical practitioner's use of the EBM guidelines. For example, the Program's website tracks the access time for each webpage reviewed, if the time of usage for each page does not meet a predetermined minimum, then the medical practitioner may be questioned concerning the legitimate usage of the EBM guidelines. However, the predetermined minimum time period for accessing a webpage is not a rigid requirement. Rather, the minimum access time period may vary from practitioner to practitioner and from diagnosis to diagnosis based on various parameters such as but not limited to the medical practitioner's area of expertise and experience and whether a particular webpage has been previously reviewed and/or printed by the medical practitioner.

In another aspect, the website provides the means for monitoring the frequency of treatments outside of the EBM guidelines (8 and 8b). Thus, the current invention provides health plans using the methods of the current invention with the ability to audit medical practitioners who may not be using the best treatments for their patients by using treatments outside of generally accepted procedures. As indicated above the methods of the current invention are flexible and can be adjusted for individual medical practitioners on the basis of their practice area and experience.

Preferably, the medical practitioner accesses the website from time to time to obtain the current EBM guidelines for diagnoses common to the medical practitioner's field. Thus, the website provides an additional source of reference and education for the medical practitioner.

In instances where the medical practitioner's diagnosis does not correspond to a diagnosis contained in the EBM data base (9), then a notification to access the Program's website will not be sent to the medical practitioner. Under these conditions, the Health Plan will direct the payor to compensate the medical practitioner at an intermediate rate. Preferably, the medical practitioner will continue to file claims for compensation via email or other electronic means even when a guideline does not exist for a specific diagnosis. As noted above, an electronically filed claim is “read” by a computer. When the computer does not find a diagnosis code corresponding to a medical diagnosis in the Program's data base, the computer will automatically direct compensation to be paid according to an intermediate rate scale.

The foregoing steps of the method of the current invention provide an incentive to the medical practitioner to comply with the treatments specified in the EBM guideline data base and to rate patient compliance with prescribed treatment/lifestyle necessary to manage the medical condition. The treatments specified in the EBM data base are the preferred treatments as determined by leading medical schools in the United States. In particular, the following schools conduct rigorous reviews of medical conditions and provide guidelines for treatments generally accepted by medical practitioners as the preferred treatments for the identified medical conditions. Schools currently developing preferred treatment guidelines include: Duke, Vanderbilt, Emory, and Oregon Health and Science University.

Providing an incentive to the medical practitioner addresses only one part of the Total Cost of Health Care formula discussed above. In order to further improve the patient's clinical outcome and enhance health care cost control, the patient must also play a role. Accordingly, the methods of the current invention provide an incentive to the patient to take a pro-active approach to recovery from and prevention of medical conditions.

With reference now to FIG. 2, the method of the current invention provides the medical practitioner with the option of prescribing Ix for the patient (10). In the preferred embodiment, the method encourages the medical practitioner to prescribe Ix for the patient by rewarding the medical practitioner with a higher rate of compensation. Preferably, the medical practitioner will prescribe the Ix Therapy at the same time the medical practitioner is responding to the website's inquiry regarding medical practitioner's compliance with EBM guidelines for the prescribed medical treatment. The prescribed Ix will normally be provided via an Internet website. For the remainder of this discussion, the source for the prescribed Ix will be referred to as the Program's website; however, other sources of information are within the scope of the present invention.

If the medical practitioner prescribes Ix for the patient (11), then a notice in the form of an e-mail, fax, letter or other similar communication will be sent automatically to the patient by the Program. This patient notification (14) may contain the medical information or more preferably the notice will contain the information required by the patient to gain access to the Program's website.

Upon receipt of the correspondence, the patient is expected to successfully accomplish the following tasks to earn a financial reward: 1) read the information presented to them on the patient portion of the Program Website about their health condition and recommended (EBM) care, and answer questions to demonstrate their understanding of this information; 2) indicate their compliance to the recommended (EBM) and appropriate care which may include reporting health status such as weight, blood pressure, blood sugar, and resting heart rate or may request authorization to access pharmacy records to verify that prescriptions have been filled or may request verification that the patient has successfully participated in a health assessment program or may request verification that the patient has seen a medical specialist or successfully completed other recommended therapies; 3) after acknowledging their medical practitioner's adherence or deviation from a recommended treatment and taking into consideration the information they have just read on the methods website, rate their medical practitioner's performance; and optionally, 4) elect to have their medical practitioner's rating of their compliance to the prescribed treatment and Ix included in the calculation of their financial reward. This election by the patient reinforces the Program's strategic checks and balances which are intent on the patient honestly indicating their personal compliance to recommended care, appropriate self-management and a healthy lifestyle. The patient is aware that this election will cause the Program to compare their personal health compliance responses against their medical practitioner's rating of their health compliance. If the compliance indicators between the patient and the medical practitioner match, then the Program would indicate that the patient is be eligible for an additional financial reward from their Health Plan.

With reference to FIG. 2, the patient is expected to review the medical information made available by the Program's website (15). The review of the prescribed Ix material is supplemented with a questionnaire to be completed by the patient (17). In the preferred embodiment, the Program's website also provides the means to monitor the patient's access of the website and completion of the questionnaire (16). This monitoring aspect provides the network with the means to audit patient compliance with the Ix and other treatment prescribed by their practitioner. Further, the monitoring system provides the ability to award “points” to the patient for reading the Ix, and for answering questionnaires which indicate the patients' knowledge and adherence to recommended treatments. As a means to insure compliance and prevent fraud and abuse the network can designate a minimum period of access time necessary prior to awarding a point for reviewing that section of the Ix. By requiring a minimum time period, the method of the current invention ensures that the patient performs more than a cursory review of the information provided.

Following completion of the questionnaires that tests the patient's knowledge and adherence to recommended and appropriate care, and rates their medical practitioner performance against recommended and appropriate care; the website scores the patient's answers and awards points to the patient's account on the basis of the results (18). Following scoring, the patient has the option of further reviewing the Ix and repeating the questions or answering additional questions. Thus, the current invention provides the patient with the ability to gain further knowledge of their condition while enhancing the number of points awarded to their account. Clearly, the comprehensive nature and flexibility of the Program's website provides the patient with the tools necessary to improve the clinical outcome of their treatment and to improve their overall general health. Optionally, Health Plan may elect to award patients with additional points for reviewing other medical information intended to improve health and control cost available through the website.

Upon completion of the Ix and indication of adherence and understanding of recommended and appropriate care, and the rating of their medical practitioner's performance; the patient is provided with a means for notifying the Health Plan of the receipt and review of the Ix material (19). Additionally, the patient will be provided with the option of sharing the medical practitioner's rating of patient compliance with the Health Plan. Typically, the patient will be provided with separate option boxes or other “clickable” devices on the website to indicate the patient's desire to share the medical practitioner's compliance rating and to transmit a notice of completion of the Ix material to the Health Plan and/or employer. In the preferred embodiment the Program website transmits the patient's actual responses to the questionnaire completed in step 18 to the medical practitioner.

In view of the incentives offered by the method of the current invention, the patient will likely request transmission of such notices to the health plan and/or employer (20). Upon receipt of such notices, the Health Plan has the option of providing a financial reward to the patient based on the patient's completion of the Ix material, rating of their practitioner, and the patient's compliance rating as provided by the medical practitioner. In keeping with the flexible nature of the current invention, the financial reward may be granted upon the completion of each prescribed Ix and practitioner rating portion. Alternatively, the party paying the reward may establish point thresholds for payouts. In the case of point thresholds, the patient's points are accumulated and upon reaching a predetermined level the financial reward can be paid to the patient.

As noted above, the method of the current invention preferably includes the medical practitioner's confirmation of the patient's application of the Ix therapy and patient compliance with the prescribed treatment/lifestyle necessary to manage the medical condition. In the preferred embodiment, point awards are conditioned upon the patient practicing the knowledge gained through Ix and making lifestyle changes prescribed by the medical practitioner. Accordingly, if the patient has been diagnosed with heart disease and has indicated full treatment adherence but has not taken preventive measures indicated by the Ix such as exercising and quitting smoking, then when the medical practitioner completes the patient rating portion of the medical practitioner's website, the medical practitioner will either indicate Non-compliant or Partially Compliant or No Response. The network would then have the option of deducting points as well as adjusting the patient's co-pay and/or deductible and/or taking other more severe steps.

Finally, the current invention also preferably provides for patient inquiries of the medical practitioner through the website, by e-mail or other similar means, during the Ix. Thus, the current invention integrates the patient's Ix with the medical practitioner's medical treatment and provides financial rewards to the patient based on completing the educational aspects of Ix as well as financial rewards for practicing a healthy lifestyle and adherence to treatment protocols as directed by the medical practitioner, and for rating their practitioner performance against recommended and appropriate care.

In accordance with the Health Insurance Portability and Accountability Act, the notice to the Health Plan and any notices to any other third parties will not divulge any protected patient health information.

Clearly, the method of the current invention provides an incentive to the patient to take an active role in managing their medical condition. As a result, the clinical outcome of the patient's medical treatment will be enhanced. Thus, the methods of the current invention enhance the quality of medical care by encouraging the patient and medical practitioner through financial rewards and profound check and balances to adhere to the scientifically proven best treatment guidelines or preferred methods and by enabling the patient to manage the treatment of the medical condition. By enhancing the quality of medical care and increasing the patient's ability to manage their medical condition, the current invention reduces the overall costs of health care while providing an increase in compensation to the medical practitioner.

Other embodiments of the current invention will be apparent to those skilled in the art from a consideration of this specification or practice of the invention disclosed herein. However, the foregoing specification is considered merely exemplary of the current invention with the true scope and spirit of the invention being indicated by the following claims.

I claim:

1. A method for delivering health care services comprising the steps of:
receiving a claim for compensation for medical services from a medical practitioner for medical treatment of a patient, said claim including at least one diagnosis code corresponding to at least one medical treatment received by said patient;
determining if at least one diagnosis code corresponds to a medical diagnosis found in a data base of medical diagnoses;
sending a notice to the medical practitioner when at least one diagnosis code submitted by said medical practitioner corresponds to a medical diagnosis found in said data base;
providing the medical practitioner access to a website, said website provides the medical practitioner with access to EBM treatment guidelines for said medical diagnosis;
said medical practitioner prescribes information therapy for said patient;
said medical practitioner provides said patient with instructions relating the medical diagnosis and a healthy lifestyle; and,
said practitioner rating patient compliance with the prescribed information therapy and instructions relating to a healthy lifestyle.

2. A method for delivering health care services comprising the steps of:
receiving a claim for compensation for medical services from a medical practitioner for medical treatment of a patient, said claim including at least one diagnosis code corresponding to at least one medical treatment received by said patient;
determining if at least one diagnosis code corresponds to a medical diagnosis found in a data base of medical diagnoses;
sending a notice to the medical practitioner when at least one diagnosis code submitted by said medical practitioner corresponds to a medical diagnosis found in said data base;
providing the medical practitioner access to a website, said website provides the medical practitioner with access to EBM treatment guidelines for said medical diagnosis;
said medical practitioner prescribes information therapy for said patient, said information therapy includes guidelines for healthy behavior related to said medical treatment;
said practitioner rating patient compliance with the prescribed information therapy;
following prescription of information therapy, said website automatically generates a notice for transmission to said patient directing said patient to access said website; and,
following the entry of a compliance rating by said medical practitioner, said website notifies said patient and provides said patient with the option of forwarding the compliance rating to the patient's Health Plan and/or employer.

3. A method for delivering health care services comprising the steps of:
receiving a claim for compensation for medical services from a medical practitioner for medical treatment of a patient, said claim including at least one diagnosis code corresponding to at least one medical treatment received by said patient;
determining if at least one diagnosis code corresponds to a medical diagnosis found in a data base of medical diagnoses;
sending a notice to the medical practitioner when at least one diagnosis code submitted by said medical practitioner corresponds to a medical diagnosis found in said data base;
providing the medical practitioner access to a website, said website provides the medical practitioner with access to EBM treatment guidelines for said medical diagnosis;
said medical practitioner prescribes information therapy for said patient, said information therapy includes guidelines for healthy behavior related to said medical treatment;
said practitioner rating patient compliance with the prescribed information therapy;
following prescription of information therapy, said website automatically generates a notice for transmission to said patient directing said patient to access said website;
said website providing said patient with the means to access medical information relating to said medical diagnosis;
monitoring the access of said medical information by said patient;
providing said patient with a knowledge exam to determine said patient's understanding of the medical diagnosis;
automatically scoring said knowledge exam;
providing said patient with the option of forwarding the knowledge exam results to patient's Health Plan; and,
providing said patient with the option of forwarding the compliance rating to the patient's Health Plan and/or employer.

4. A system for delivering health care services comprising:
a website having a medical practitioner portion and a patient portion;
said medical practitioner's portion being programmed to receive a coded claim for medical services rendered by said medical practitioner, comparing said coded claim to a database of medical diagnoses, if said coded claim matches a medical diagnosis in said database, then said website sends a notice to said medical practitioner directing the medical practitioner to access said website;
said medical practitioner portion of said website includes a database of EBM guidelines, patient names, dates of services provided and medical diagnoses;
said website provides for input of medical practitioner compliance with EBM guidelines;
said website provides for input by said medical practitioner of a patient's compliance with prescribed treatment;
said website provides for tracking of medical practitioner during access of said website;
said patient portion of said website includes a database accessible by said patient;
said patient portion of said website provides said patient with a knowledge exam;
said patient portion of said website provides the means for forwarding patient results of said knowledge exam to a third party;
said patient portion of said website provides the means for forwarding the input provided by said medical practitioner of patient's compliance with prescribed treatment to a third party.
